(19) United States
(12) Patent Application Publication
(10) Pub. No.: US 2026/0207443 A1
(43) Pub. Date: Jul. 23, 2026

(54) CREAM HAIR CLEANING COMPOSITION

(71) Applicant: Church & Dwight Co., Inc., Princeton, NJ (US)

(72) Inventors: Amie Stewart, Denville, NJ (US); Sophie Walsh, Kent (GB)

(73) Assignee: Church & Dwight Co., Inc., Princeton, NJ (US)

(21) Appl. No.: 19/453,794

(22) Filed: Jan. 20, 2026

Related U.S. Application Data
(60) Provisional application No. 63/747,786, filed on Jan. 21, 2025.

(51) Int. Cl. A61K 8/02; A61Q 5/02

(52) U.S. Cl. CPC A61K 8/022; A61Q 5/02; A61K 2800/30; A61K 2800/49; A61K 2800/5922

(57) ABSTRACT

The present disclosure is directed to a hair cleaning composition the includes a plurality of solvents that include water and at least one volatile solvent, at least one oil-absorbent material, and at least one rheology modifier. The hair cleaning composition does not include an emollient. The hair cleaning composition is configured as a cream that is convertible to a powder upon application of mechanical force. The present disclosure is also directed to a method of cleaning hair, including the steps of dispensing an amount of the cream hair cleaning composition, mechanically activating the cream hair cleaning composition with hands or fingers to convert the cream to a powder; distributing the powder one or both of through the hair and along the scalp of the user; and removing excess powder present from the hair or scalp.

This application claims priority to and the benefit of U.S. provisional patent application No. 63/747,786, filed Jan. 21, 2025 and entitled “Cream Hair Cleaning Composition” the entire contents of which are incorporated herein by reference.

### TECHNICAL FIELD

The present disclosure relates to a no-rinse hair cleaning composition that is configured as a cream and becomes a powder upon application. This hair cleaning composition is fast drying, and effective for absorbing excess oil from hair with substantially no residue. The present disclosure is also directed to a method of cleaning hair using the no-rinse hair cleaning composition.

### BACKGROUND

A majority of no-rinse hair cleaning compositions, such as dry shampoos, are presented in aerosol formulations and offer numerous performance benefits such as ease of use, application time and control, and dry time. There is a growing market for non-aerosol no-rinse hair cleaning compositions for environmental and personal care reasons. For instance, non-aerosol no-rinse hair cleaning composition may include a loose powder formula that is applied to hair, but such formulations can be messy and may not offer the controlled application of an aerosol.

After application with an aerosol formulated dry shampoo, most consumers feel the need to brush their hair to blend in visible residue or powder that is left on the hair and scalp. Often times, aerosol formulations use white colored oil absorbent particles, which can leave a “white-cast” on darker colored hair. Brushing, combing, or blow-drying the hair after application becomes necessary to remove the “white-cast.” This makes aerosol products difficult to use for curly haired consumers who tend to only use a brush on their hair when their hair is wet because brushing their curly hair when dry leads to undesired frizz.

There remains a need for a no-rinse hair cleaning composition that blends into hair without the need to further brush or comb hair to remove excess product, has excellent oil-absorption, and leaves no unpleasant residue on the hair or scalp.

### SUMMARY

The present disclosure is directed to a no-rinse hair cleaning composition that may be configured as a cream and transforms into a powder upon application. The no-rinse hair cleaning composition blends in to a user's hair during application such that the composition leaves an “invisible finish,” meaning that no visible residue or substantially no visible residue is present on the hair. Likewise, application of the present composition does not leave a powdery, tacky, or stiff residue feeling on the hair.

In one or more embodiments, the present disclosure can relate to a hair cleaning composition that includes a plurality of solvents, at least one oil-absorbent material, and at least one rheology modifier. The plurality of solvents may include water and at least one volatile solvent.

In some embodiments, the hair cleaning composition does not include an emollient.

In some embodiments, the hair cleaning composition may be configured as a cream that is convertible to a powder upon application of mechanical force.

The at least one volatile solvent may include at least one of an alcohol and a siloxane, and the siloxane may include disiloxane.

The at least one rheology modifier may include a crosspolymer.

The at least one oil-absorbent material may include at least one starch or clay, or may include one or both of distarch phosphate and rice starch.

In some embodiments, the hair cleaning composition may include one or more materials such as fragrances, preservatives, colorants, and combinations thereof.

In some embodiments, the water may be present in an amount of about 55% to about 65% by weight, at least one volatile solvent may be present in an amount of about 15% to about 25% by weight, the at least one oil-absorbent material may be present in an amount of about 15% to about 20% by weight, and the at least one rheology modifier may be present in an amount of about 0.1% to about 0.5% by weight, where each of the foregoing is based on a total weight of the hair cleaning composition.

In some embodiments, the hair cleaning composition may be configured to be distributable through hair without the requirement of a hair styling implement.

In some embodiments, the cream may have a viscosity ranging from greater than about 20,000 cps to less than about 30,000 cps.

In some embodiments, the hair cleaning composition may be configured to be substantially completely dry within a time of 12 minutes or less after application to the hair.

In some embodiments, the cream converts to the powder upon mechanical force applied by hands or fingers within a time of 30 seconds or less.

In one or more embodiments, the present disclosure can relate to a method of cleaning hair including the steps of dispensing an amount of a cream hair cleaning composition, the hair cleaning composition may include a plurality of solvents including water and at least one volatile solvent, at least one oil-absorbent material, and at least one rheology modifier; mechanically activating the cream hair cleaning composition with hands or fingers to convert the cream to a powder; distributing the powder one or both of through the hair and along the scalp; and removing excess powder present from the hair or scalp.

In some embodiments, the step of mechanically activating the cream hair cleaning composition with hands or fingers to convert the cream to a powder may take less than 30 seconds.

In some embodiments, one or both of the following conditions is met: distributing the powder includes mechanically moving the powder with hands or fingers; and removing excess powder includes manipulating one or both of the scalp and hair with hands or fingers.

Aspects and advantages of these exemplary embodiments and other embodiments, are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a series of images of hair tresses exposed to artificial sebum, where the top row corresponds to Example 1, and the bottom row corresponds to Comparative Example 1.

FIG. 2 is a graph demonstrating the efficacy of various hair cleaning compositions on hair tresses exposed to artificial sebum.

FIG. 3 is a series of images of the performance of various hair cleaning compositions in a sebum adsorption test performed on glass plates.

FIG. 4 is a series of images demonstrating the drying time of various hair cleaning compositions using films left on a lab bench under ambient conditions, and imaged at intervals of 8 minutes, 11 minutes, 12 minutes, 18 minutes, and 22 minutes.

FIG. 5 is a series of images of the performance of various hair cleaning compositions in an oil visualization test where the formulations were applied to a clear plastic sheet films and were left on a lab bench under ambient conditions for 1 hour.

FIG. 6 is a series of images of the performance of various hair cleaning compositions in an oil visualization test where the formulations were left to dry in a weight boat.

FIG. 7A is an image of the formula of Comparative Example 1 applied to applied to plastic sheet, allowed to dry and then wiped away to show the amount of residue remaining; and FIG. 7B is an image of the formula of Example 1 applied to plastic sheet, allowed to dry and then wiped away to show the amount of residue remaining.

### DETAILED DESCRIPTION

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The terms “about” or “approximately” are defined as being close to as understood by one of ordinary skill in the art. In one non-limiting embodiment, the terms are defined to be within 10%, preferably within 5%, more preferably within 1%, and most preferably within 0.5%. The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The present disclosure relates to a hair cleaning composition that includes a plurality of solvents comprising water and at least one volatile solvent, at least one oil-absorbent material, and at least one rheology modifier. The hair cleaning composition does not include an emollient, and is initially in the form of a cream that transition to a powder when rubbed between hands or fingers prior to application to hair.

Hair cleaning compositions, such as dry shampoos, are often delivered in aerosol format where a user sprays the dry shampoo from a distance away from the hair and scalp. Then, the user allows time for the dry shampoo to absorb oil, and works the product into the hair using their hands and fingers. Aerosol formulations are often quick drying and allow for controlled application to specific areas of the hair. In many cases, the aerosol may leave a white, powdery cast to the hair, which users have to remove from their hair by brushing, combing, or blow-drying. This is not ideal for all hair types, and those with curly hair may have frizz after brushing dry hair.

There are several other reasons why the use of aerosol formulations may be undesirable such as the negative effects of propellants on the environment, and difficultly in airplane travel with aerosols. Therefore, there is a need for alternative forms of no-rinse hair cleaning compositions that have superior oil-absorption, are quick drying, and are easy to use.

In one or more embodiments, the present disclosure provides a hair cleaning composition that is in the form of a cream, but transitions to a powder upon application to the hands and hair. The hair cleaning composition dries quickly, absorbs oil efficiently, and results in a refreshing feel on the hair.

Cream-to-powder hair cleaning composition is a new non-aerosol form that is applied with fingers to exactly where the product is needed on the hair and scalp. Surprisingly, many consumers noted that the cream-to-powder formulation and application method provides more control and less mess than aerosol formulations, which tends to overspray on hair, clothes, etc. and leaves a cloud of powder and fragrance. The cream-to-powder hair cleaning composition also does not require rinsing with water, or the removal of excess powder from the hair and scalp with a hair styling implement such as a hairbrush.

The creamy initial product quickly transforms into a powder when rubbed between fingers and/or onto skin. By adding a relatively low amount of volatile solvents, such as ethanol and disiloxane, to the aqueous base, the liquid media quickly evaporates, leaving only oil-absorbing powder behind. With no added oil, such as emollients, in the formulation, the oil-absorbing powders will be highly effective at adsorbing excess sebum on the hair and scalp.

The hair cleaning composition may include a plurality of solvents including water and at least one volatile solvent. The water may be included in an amount of about 55% to about 65% by weight, about 56% to about 64% by weight, about 57% to about 63% by weight, about 58% to about 62% by weight, about 59% to about 61% by weight, about 55% to about 60% by weight, about 60% to about 65% by weight, based on a total weight of the hair cleaning composition.

A volatile solvent is a liquid substance that has a high vapor pressure at a given temperature, which means it easily evaporates into the air. Volatile solvents may be suitable for use in a hair cleaning composition due to their ability to dissolve other substances and then evaporate quickly, leaving behind the dissolved material, such as an oil-absorbent material. Volatile solvents may act as carriers for active ingredients, such as starches or silica, which help absorb oils and grease from the hair. These solvents help distribute these active ingredients evenly throughout the hair and then evaporate quickly, leaving the hair looking cleaner and without residue. The volatile solvents may generally have a low boiling point and high vapor pressure, which contribute to the volatility. In general, combinations of water and alcohol may create an azeotrope, which results in a lower boiling point. For instance, the boiling point may range from about 65° C. to about 110° C., and the vapor pressure at 20° C. (68° F.) may range from about 50 mmHg to about 130 mmHg.

The at least one volatile solvent may include at least one of an alcohol and a siloxane. The alcohol may include ethanol. The siloxane may include a disiloxane, which may specifically include hexamethyldisiloxane (HMDSO). The total amount of volatile solvent may be included in an amount of about 10% to about 30%, about 15% to about 25% by weight, about 16% to about 24% by weight, about 17% to about 23% by weight, about 18% to about 22% by weight, or about 19% to about 21% by weight, based on a total weight of the hair cleaning composition.

When both an alcohol and a disiloxane are present in the composition as volatile solvents, the alcohol may be present in an amount of about 5% to about 20%, about 10% by weight to about 15% by weight, about 11% by weight to about 14% by weight, about 12% by weight to about 13% by weight, about 10% by weight to about 12.5% by weight, or about 12.5% by weight to about 15% by weight, based on a total weight of the hair cleaning composition. The disiloxane may be present in an amount of about 2% to about 12%, about 5% by weight to about 8% by weight, about 5% by weight to about 7.5% by weight, about 5% by weight to about 7% by weight, about 5% by weight to about 6.5% by weight, about 5% by weight to about 6% by weight, about 5.5% by weight to about 7.5% by weight, about 6% by weight to about 7.5% by weight, about 6.5% by weight to about 7.5% by weight, or about 7% by weight to about 7.5% by weight, based on a total weight of the hair cleaning composition.

The hair cleaning composition may include an oil-absorbent material or oil-absorbent powder useful for removing or absorbing excess oil, grease, and other undesirable elements from hair. For example, the hair cleaning composition may include at least one clay material or at least one starch material that is effective to absorb at least a portion of any oils present on hair. Clay materials in the present disclosure may include kaolin, and starch materials may include distarch phosphate, rice starch, cornstarch, potato starch, tapioca starch, wheat starch, cassava starch, and combinations thereof. In certain embodiments, the hair cleaning composition may include kaolin. A starch material can be modified (e.g., through processes such as esterification, etherification, oxidation, acid hydrolysis, crosslinking, or enzyme conversion) or unmodified. In some embodiments, a starch may be expressly excluded or the clay may be the only cleaning agent used in the hair cleaning composition.

In some embodiments of the present disclosure, the weight percentage of the oil-absorbent material in the hair cleaning composition, based on the total weight of the hair cleaning composition, can be about 15% to about 20% by weight, about 16% to about 19% by weight, or about 15% to about 18% by weight. In certain embodiments, the weight percentage of the oil-absorbent material in the hair cleaning composition, based on the total weight of the hair cleaning composition, can be at least about 15%, at least about 16%, at least about 17%, at least about 18%, or at least about 19%, preferably with an upper range limit of about 20% by weight.

The hair cleaning composition may include at least one rheology modifier including a crosspolymer. The crosspolymers may include acrylate-based crosspolymers, such as acrylates/vinyl isodecanoate crosspolymer (STABYLEN™ 30) and acrylates/C10-30 alkyl acrylate crosspolymer (CARBOPOL® ULTREZ-21) may be included as the rheology modifier. A rheology modifier is an ingredient used to adjust the viscosity and flow properties of a liquid, such as how thick or thin a product is, which can affect both its performance and user experience. The rheology modifier is present to provide additional stability to the aqueous suspension, and to prevent separation of the aqueous phase and the oil-absorbent powder. In addition, a caustic soda such as sodium hydroxide may also be present in order to neutralize the presence of the acidic crosspolymers. In certain embodiments, the weight percentage of the rheology modifier can be at least about 0.1% to about 0.5% by weight, about 0.2% to about 0.4% by weight, about 0.2% to about 0.5% by weight, about 0.1% to about 0.4% by weight, based on a total weight of the hair cleaning composition.

In addition, other materials that benefit the scalp and/or hair may be included, such as water-soluble actives including fragrances, preservatives, colorants, and combinations thereof. For example, the preservatives may include phenols such as phenoxyethanol, and are used in cosmetics to prevent the growth of harmful bacteria and mold. For example, the colorants may include a blonde colorant, a brunette colorant, a red colorant, a light colorant, or a dark colorant.

The cream converts, or transitions, to a powder upon mechanical force, manipulation or deformation applied to the cream by hands or fingers. The cream may be dispensed on to the hands, and then mechanical force can be applied prior to application to the hair. The mechanical force may include rubbing or pressing fingers or hands together. The cream converts to powder upon mechanical force applied by hands or fingers within a time of about 30 seconds or less, about 25 seconds or less, about 20 seconds or less, about 15 seconds or less, about 10 seconds or less, or about 5 seconds or less.

The hair cleaning composition may be characterized as an aqueous suspension. An aqueous suspension is a type of mixture in which solid particles are dispersed throughout an aqueous, liquid medium, which may be, for example, water or a mixture of water with one or more further liquids that are stably combinable with the water. In an aqueous suspension, the solid particles do not dissolve in the water but remain suspended within it. The stability of an aqueous suspension can be enhanced with the use of stabilizing agents or thickeners, such as rheology modifiers, that prevent the solid particles from settling. The solid particles may be unsuspended from an aqueous suspension using mechanical force, which may involve agitation or the application of shear forces to overcome the interactions keeping the particles suspended.

This is different from an emulsion in which two immiscible liquids, such as oil and water, are combined where one liquid is dispersed in the form of droplets within the other. Emulsions often require an emulsifying agent, such as a surfactant, to stabilize the mixture.

In a present embodiment, the oil-absorbent material is suspended in the aqueous solvent. Upon mechanical force, manipulation, or deformation applied to the cream by hands or fingers, the oil-absorbent material is released from the aqueous suspension to form a powder by spreading or rubbing. The aqueous suspension may release the oil-absorbent through a transformation process including the evaporation of volatile solvents and the physical action of applying the product to the hands or fingers and rubbing the hands or fingers together.

By not including any additional oils or emollients in the hair cleaning composition, the oil-absorbent material is able to more freely adsorb to bodily oils, such as sebum, present on the hair and scalp. If additional oils or emollients are present in the hair cleaning composition, the oil-absorbent material may not adsorb as effectively to bodily oils, and instead may bind to the additional oils or emollients present in the hair cleaning composition.

The hair cleaning composition may be configured to be distributable through hair without requirement of a hair styling implement. For instance, a user can apply the cream-to-powder formulation to the hair with only the use of hands or fingers, and does not require brushing, combing, diffusing, or blow-drying to remove excess product from the hair.

The initial cream of the hair cleaning composition may have a viscosity of less than 30,000 centipoise (cps). For example, viscosity may range from about 20,000 cps to about 30,000 cps, from about 21,000 cps to about 29,000 cps, from about 22,000 cps to about 28,000 cps, from about 23,000 cps to about 27,000 cps, from about 24,000 cps to about 26,000 cps, from about 20,000 cps to less than about 25,000 cps, and from about 25,000 cps to less than about 30,000 cps. The viscosity ranges are measurable using, for example, a Brookfield DVPlus Viscometer equipped with an LV64 spindle and operated at a sheer rate of about 5 RPM.

The hair cleaning composition is configured to be substantially completely dry within a time of about 12 minutes or less after application to the hair. In one or more embodiments, the composition may be configured to be substantially completely dry, meaning the hair does not feel wet when touched by hands and does not appear wet on visual inspection, within a time of about 10 minutes or less, about 8 minutes or less, about 5 minutes or less, about 3 minutes or less, or about 2 minutes or less, such as about 15 seconds to about 15 minutes, about 30 seconds to about 10 minutes, about 45 seconds to about 5 minutes, or about 1 minute to about 3 minutes. Substantially none or no powdery residue remains after the application of the cream-to-powder formulation is applied to hair.

The hair cleaning composition may be configured for delivery in an airless pump-style container having a mechanical pump, or other container effective for dispensing the formulation in the initial cream state such as a tube, tub, jar, single-use sachet, or compact. A pump-style container may be preferred to control delivery of the hair cleaning composition into the user's hands.

Another embodiment includes a method of cleaning hair using the hair cleaning composition. A consumer may dispense an amount of the hair cleaning composition as a cream into their hands, and rub their hands or fingers together to mechanically disperse or activate the cream. A consumer may apply the hair cleaning composition by contacting hair with the powder. Upon contact with the hair, the oil-absorbent material of the hair cleaning composition may be deposited on the hair by mechanically moving the hair cleaning composition through the hair. This step of distributing or depositing the powder on the hair may include rubbing the hair with hands or fingers. The hair may be tousled to distribute the oil-absorbent material throughout the hair to absorb oil from the hair. No rinsing with water is required to apply the hair cleaning composition, or to deposit the oil-absorbent material onto the hair.

Next, any excess of the powder present on the hair may be removed by further mechanical action such as rubbing, tousling, or manipulating one or both of the scalp and hair with hands or fingers. Manipulating may include any kind of movement using hands, fingers, or even merely shaking the hair. Removal specifically can be carried out in the absence of water rinsing, and without the need to brush or comb the hair with a hair styling implement.

### EXAMPLES

The following are examples and comparative examples of hair cleaning compositions prepared as cream formulations.

The following Table 1 provides the formulation of the hair cleaning composition of Examples 1 to 3. The components and weight percentages of each example are shown in Table 1.

TABLE 1

| | | Exam- | Exam- | Exam- |
|---|---|---|---|---|
| | | ple 1 | ple 2 | ple 3 |
| | Component | % w/w | % w/w | % w/w |
| WATER | Aqua | 60.10 | 56.10 | 65.10 |
| RICE PO4 | Distarch Phosphate | 12.50 | 14.00 | 12.50 |
| NATURAL | | | | |
| ETHANOL DEB96 | Denatured Alcohol | 12.50 | 15.00 | 10.00 |
| RICE STARCH | Oryza Sativa (Rice) | 5.00 | 5.00 | 5.00 |
| D.S.A. 7 | Starch (and) | | | |
| | Cetrimonium | | | |
| | Chloride | | | |
| CARBOPOL ® | Acrylates/C₁₀₋₃₀ Alkyl | 0.10 | 0.10 | 0.10 |
| ULTREZ-21 | Acrylate | | | |
| | Crosspolymer | | | |
| STABYLEN ™ 30 | Acrylates/Vinyl | 0.30 | 0.30 | 0.30 |
| | Isodecanoate | | | |
| | Crosspolymer | | | |
| VOLASIL ® DM- | Disiloxane | 7.50 | 7.50 | 5.00 |
| 0.65 | | | | |
| EUXYL ™ PE9010 | Phenoxyethanol & | 1.00 | 1.00 | 1.00 |
| | Ethylhexylglycerin | | | |
| ALOE VERA 200:1 | Aloe Barbadensis | 0.10 | 0.10 | 0.10 |
| | (Aloe Vera) Leaf | | | |
| | Juice | | | |
| FRAGRANCE | GIVAUDAN Green | 0.50 | 0.50 | 0.50 |
| | Smoke UAJ13004/00 | | | |
| CAUSTIC SODA | Sodium Hydroxide | 0.40 | 0.40 | 0.40 |
| 32% | | | | |

| | | | |
|---|---|---|---|
| Total: | 100.00 | 100.00 | 100.00 |

### COMPARATIVE EXAMPLES

Several formulations were tested as Comparative Examples. Each of the comparative Examples corresponds to a marketed product.

Comparative Example 1 corresponds to a formula of Raw® Sugar Not So Dry Shampoo and includes water (aqua), tapioca starch, stearyl alcohol, behentrimonium chloride, cetyl alcohol, glycerin, aloe barbadensis leaf juice (certified organic/cold press), Bambusa arundinacea stem extract, Leuconostoc/radish root ferment filtrate, Phaseolus angularis seed extract (certified organic/cold press), biotin, Avena sativa (oat) kernel flour, Argania spinosa kernel oil (certified organic/cold press), coconut alkanes, coco-caprylate/caprate, levulinic acid, sodium levulinate, citric acid, sodium hydroxide, dehydroacetic acid, benzyl alcohol, phenoxyethanol, and fragrance (parfum).

Comparative Example 2 corresponds to a formula of R+Co® Vapor Lotion to Powder Dry Shampoo and includes water, tapioca starch, cyclopentasiloxane, glycerin, Maranta arundinacea root powder, cetearyl alcohol, cetearyl olivate, phenoxyethanol, sorbitan olivate, fragrance (parfum), silica, dimethicone crosspolymer, potassium sorbate, ethylhexylglycerin, citric acid, ethyl linoleate, ethyl oleate, cyclodextrin, ethylhexyl methoxycinnamate, Persea gratissima (avocado) oil, colloidal oatmeal, Crambe abyssinica seed oil, Helianthus annuus (sunflower) seed oil, benzyl alcohol, caprylyl/capryl glucoside, caprylic acid, guar hydroxypropyltrimonium chloride, xylitol, Leuconostoc/radish root ferment filtrate, tocopherol, limonene, benzyl salicylate, linalool, and citral.

Comparative Example 3 corresponds to a formula of Alterna® Meltaway and includes water, denatured alcohol, tapioca starch, caprylyl methicone, phenoxyethanol, polymethylsilsesquioxane, polyacrylate-13, fragrance, polyisobutene, silicone quaternium-16/glycidoxy dimethicone crosspolymer, limonene, ethylhexylglycerin, polysorbate 20, undeceth-11, sorbitan isostearate, linalool, propylene glycol, undeceth-5, glycerin, acetic acid, charcoal powder, tocopherol, polyaminopropyl biguanide, Caulerpa lentillifera extract, citric acid, sodium benzoate, sodium hydroxide, glucose, sodium chloride, hydrolyzed pea protein, and sodium succinate.

Comparative Example 4 corresponds to CIBU® Dry Shampoo Cream and includes water, tapioca starch, cyclopentasiloxane, cetearyl alcohol, glycerin, Maranta arundinacea root powder, cetearyl olivate, caprylyl/capryl glucoside, phenoxyethanol, sorbitan olivate, dimethicone crosspolymer, guar hydroxypropyltrimonium chloride, fragrance, Amaranthus caudatus seed extract, Coffea arabica (coffee) seed extract, Oryza sativa (rice) extract, ethyl linoleate, ethyl oleate, Crambe abyssinica seed oil, Helianthus annuus (sunflower) seed oil, Persea gratissima (avocado) oil, Avena sativa (oat) kernel flour, xylitol, Leuconostoc/radish root ferment filtrate, silica, tocopherol, hydroxycitronellal, benzyl benzoate, coumarin, sorbic acid, potassium sorbate, sodium benzoate, and ethylhexylglycerin.

Comparative Example 5 corresponds to an aerosol-type of dry shampoo formulation, Batiste Fresh Scent®, rather than a cream-to-powder formulation. The formulation includes butane, isobutane, propane, Oryza sativa (rice) starch, denatured alcohol, parfum (fragrance), cocoyl hydrolyzed keratin, cetrimonium chloride, and distearyldimonium chloride.

The viscosity of the Examples and Comparative Examples was measured using a Brookfield DVPlus Viscometer using an LV64 spindle, and a sheer rate of 5 RPM. The viscosity of each initial cream is listed in Table 2 below.

TABLE 2

| Product | Viscosity |
|---|---|
| Example 1 | 29,160 cps (centipoise) |
| Example 2 | 23,312 cps |
| Example 3 | 27,310 cps |
| Comparative Example 1 | 30,240 cps |
| Comparative Example 2 | 40,560 cps |
| Comparative Example 3 | 67,680 cps |
| Comparative Example 4 | 48,960 cps |

### Sebum Adsorption Test-Hair Tress Test

Blonde hair tresses were treated with artificial sebum. The sebum was dyed red for visualization purposes. Examples 1-3 and Comparative Examples 1-3 were visually compared. While Example 2 had higher sebum adsorption in vitro, the lower viscosity of the formula was less preferred by the consumer.

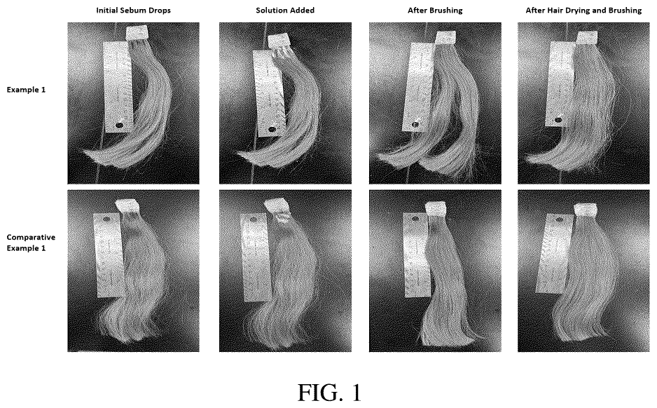

FIG. 1 shows the results of the experiments. Two drops of artificial sebum (dyed red) were applied to a hair tress. The length of the sebum was measured, and a photo was taken to document the initial state. For each experiment, one pump of cream-to-powder formulation was then applied to the hair tress, rubbed in from top to bottom and subsequently photographed. The hair tress was brushed thoroughly, measured again, and photographed. Finally, a blow dryer was used on low heat for 30 seconds on the hair tress, and the length was measured and photographed.

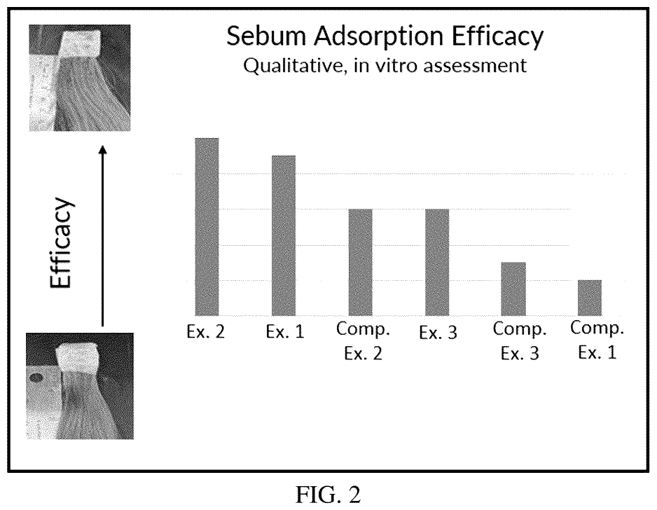

FIG. 2 graphically shows the sebum adsorption efficacy of Examples 1-3 and Comparative Examples 1-3. As shown, Example 2 achieves the highest sebum adsorption efficacy, followed by Example 1.

### Sebum Adsorption Test on Glass Plate

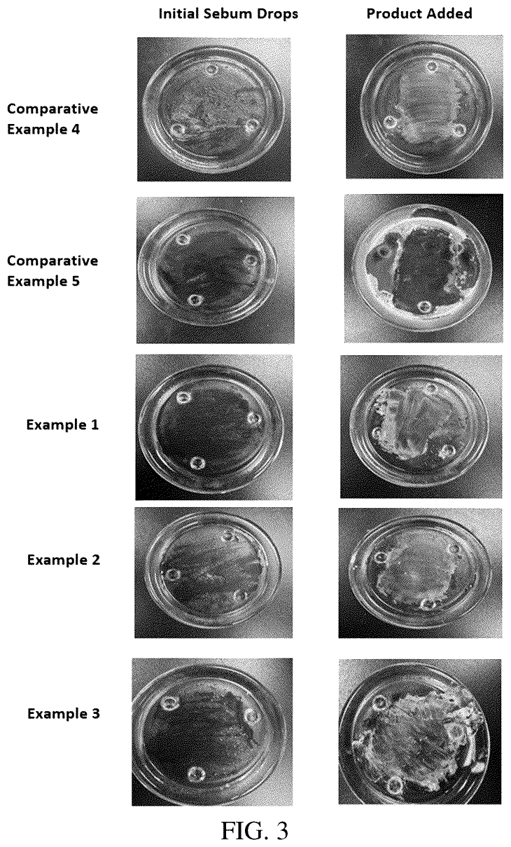

The ability of the formulations of Examples 1-3 and Comparative Examples 4 and 5 to absorb sebum were compared. Three drops of artificial sebum (dyed red) were spread onto each glass plate. As shown in FIG. 3, a photograph was taken to capture the initial condition of the formulas on the glass plates. For each experiment, one pump of cream-to-powder formulation was spread on a glass plate, and after waiting 2 minutes for it to dry, the plate was wiped with two fingers and photographed. The relative difference in the amount of sebum that is bound by the powders was visually observed. In the higher oil-absorbing formulas of Examples 1-3, the sebum was removed from the glass. In the lower oil-absorbing formulas of Comparative Examples 4 and 5, the oily residue remained on the glass.

### Drying Time

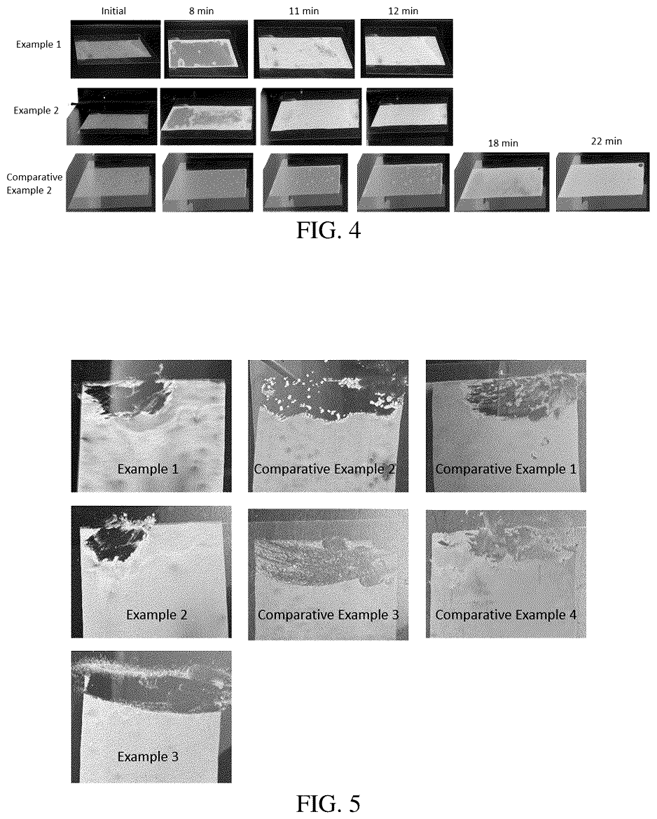

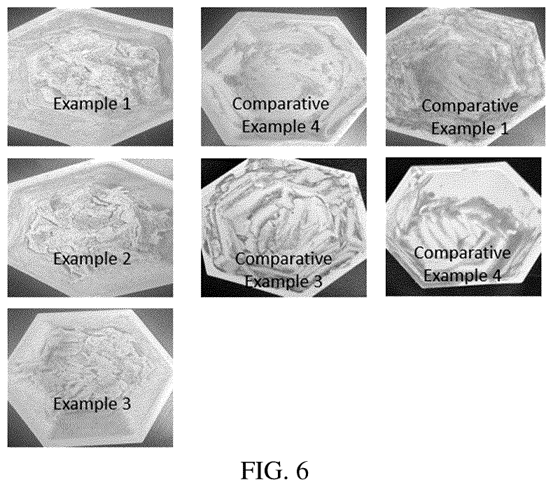

The time for drying for each formula of Examples 1 and 2, and Comparative Example 2 were compared. Each formula was spread on a clear plastic sheet using a square applicator to provide a drawdown film of 2 mL. The films were left on a lab bench under ambient conditions. The films were monitored and the time to dry was recorded as show in Table 3 below and in FIG. 4. The dry time is expected to be less when applied to hair than in the experimental tests performed on films.

TABLE 3

| | Dry time (min) |
|---|---|
| Example 1 | 12 |
| Example 2 | 12 |
| Comparative Example 1 | 42 |
| Comparative Example 2 | 22 |
| Comparative Example 3 | 15 |
| Comparative Example 4 | 17 |

### Detection and Visualization of Oil

The Examples 1-3 are formulated without oil, which is an important distinction from the Comparative Examples 1-4. The cream-to-powder formulations of Examples 1-3 contain starch powder that can adsorb sebum to give a consumer the look of clean, oil-free hair. The Comparative Example formulations contain oil, which can (1) add to the appearance of oily/greasy roots, (2) bind to the starch, making it less available to adsorb sebum, and/or (3) leave a residue on the hair/scalp.

To show the oil content, Oil Red O, was used as an oil soluble dye. The dye was dissolved into a homogeneous, red-colored solution in the presence of oil. When combined with water, the dye did not dissolve, and there was a heterogenous mixture. As shown in FIG. 5, Examples 1-3 and Comparative example 1-4 were compared. Each product (6.0 g) was combined with 0.005 g of Oil Red O and mixed thoroughly. Each formula was spread on a clear plastic sheet using a square applicator to provide a drawdown film of 2 mL. The films were left on a lab bench under ambient conditions for 1 hour. The remaining formula-dye mixtures were left to dry in a weight boat.

In Examples 1-3, the red dye did not disperse into the film, and spots of dye in the dried cream that were not homogeneously distributed into the cream were observed because it the oil was insoluble. For Comparative Examples 1-4, the dye tinted the film pink due to the presence of oil in the Comparative formulations. The oil-soluble red dye was able to dissolve into the oil-containing Comparative Example formulations at various degrees based on the observed dried product remaining.

### Detection and Quantification of Residue

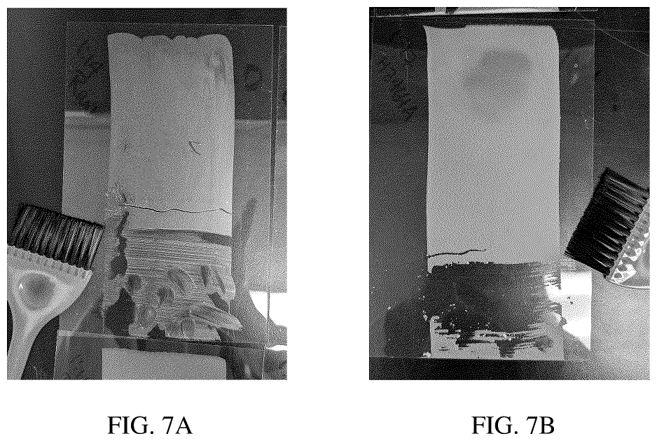

Examples 1-3 exhibit the least amount of residue as each dries down to a fine powder, whereas the formulas of the Comparative Examples dry down to a powdery mixture that feels greasy and/or waxy and leaves substantial residue. The amount of residue that the product leaves on hair and/or scalp can be seen in FIGS. 7A and 7B. Comparative Example 1 is shown in FIG. 7A, and Example 1 is shown in FIG. 7B. Each formulation was applied to a clear plastic sheet and allowed to dry. Once the formulas were dried, a small brush was used to swipe across the dried formula. As shown, Comparative Example 1 in FIG. 7A had an oily residue remaining on the sheet, whereas Example 1 in FIG. 7B exhibited no or very little residue.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A hair cleaning composition, comprising:
a plurality of solvents comprising water and at least one volatile solvent;
at least one oil-absorbent material; and
at least one rheology modifier,
wherein:
the hair cleaning composition does not include an emollient,
the hair cleaning composition is configured as a cream that is convertible to a powder upon application of mechanical force.

2. The hair cleaning composition of claim 1, wherein the at least one volatile solvent comprises at least one of an alcohol and a siloxane.

3. The hair cleaning composition of claim 2, wherein the siloxane is disiloxane.

4. The hair cleaning composition of claim 1, wherein the at least one rheology modifier comprises a crosspolymer.

5. The hair cleaning composition of claim 1, wherein the at least one oil-absorbent material comprises at least one starch or clay.

6. The hair cleaning composition of claim 5, wherein the at least one oil-absorbent material includes one or both of distarch phosphate and rice starch.

7. The hair cleaning composition of claim 1, further comprising one or more materials selected from the group consisting of fragrances, preservatives, colorants, and combinations thereof.

8. The hair cleaning composition of claim 1, wherein one or more of the following applies:
the water is present in an amount of about 55% to about 65% by weight;
the at least one volatile solvent is present in an amount of about 15% to about 25% by weight;
the at least one oil-absorbent material is present in an amount of about 15% to about 20% by weight; and
the at least one rheology modifier is present in an amount of about 0.1% to about 0.5% by weight;
each of the foregoing is based on a total weight of the hair cleaning composition.

9. The hair cleaning composition of claim 1, wherein the hair cleaning composition is configured to be distributable through hair without requirement of a hair styling implement.

10. The hair cleaning composition of claim 1, wherein the cream has a viscosity ranging from greater than about 20,000 cps to less than about 30,000 cps.

11. The hair cleaning composition of claim 1, wherein the hair cleaning composition is configured to be substantially completely dry within a time of about 12 minutes or less after application to the hair.

12. The hair cleaning composition of claim 1, wherein the cream converts to the powder upon mechanical force applied by hands or fingers.

13. The hair cleaning composition of claim 1, wherein the cream converts to the powder upon mechanical force applied by hands or fingers within a time of about 30 seconds or less.

14. A method of cleaning hair, comprising the steps of:
dispensing an amount of a cream hair cleaning composition, the hair cleaning composition comprising a plurality of solvents including water and at least one volatile solvent, at least one oil-absorbent material, and at least one rheology modifier;
mechanically activating the cream hair cleaning composition with hands or fingers to convert the cream to a powder;
distributing the powder one or both of through the hair and along the scalp of the user; and
removing excess powder present from the hair or scalp.

15. The method of cleaning hair of claim 14, wherein one or both of the following conditions is met:
distributing the powder comprises mechanically moving the powder with hands or fingers; and
removing excess powder comprises manipulating one or both of the scalp and hair with hands or fingers.

16. The method of cleaning hair of claim 14,
wherein the hair cleaning composition does not include an emollient.

17. The method of cleaning hair of claim 14, wherein the step of removing excess powder does not require brushing or combing with a hair styling implement.

18. The method of cleaning hair of claim 14, wherein the hair cleaning composition configured as the cream has a viscosity ranging from greater than about 20,000 cps to less than about 30,000 cps.

19. The method of cleaning hair of claim 14, wherein the hair cleaning composition is configured to be substantially completely dry within a time of about 12 minutes or less after application to the hair.

20. The method of cleaning hair of claim 14, wherein the step of mechanically activating the cream hair cleaning composition with hands or fingers to convert the cream to a powder is less than about 30 seconds.
